(19) United States
(12) Patent Application Publication
(10) Pub. No.: US 2018/0078171 A1
(43) Pub. Date: Mar. 22, 2018

(54) ECHOLOCATION SYSTEM AND METHOD FOR LOCATING A CATHETER TIP

(71) Applicant: MEDICAL COMPONENTS, INC., Harleysville, PA (US)

(72) Inventor: Andres B. Cruz, JR., Red Hill, PA (US)

(21) Appl. No.: 15/704,580

(22) Filed: Sep. 14, 2017

Related U.S. Application Data
(60) Provisional application No. 62/396,443, filed on Sep. 19, 2016.

(51) Int. Cl. A61B 5/06; A61B 5/00

(52) U.S. Cl. CPC A61B 5/065; A61B 5/742; A61B 5/6852

(57) ABSTRACT

A system for locating the tip of a catheter inside a human body is provided. The system includes a housing; a sound emitter and a sound sensor housed in the housing; a waveform generator configured to generate a fixed waveform at a desired frequency and having desired characteristics and output the fixed waveform to the sound emitter; a peripherally inserted central catheter fluidly coupled to the housing and configured to propagate the fixed waveform toward a heart of a patient; a sound sensor housed within said housing, said sound configured to sense sound waves reflected from the heart as the peripherally inserted central catheter is progressed toward a heart of a patient; and a waveform analyzer operably coupled to the output of the waveform generator and input from the sound sensor, the waveform analyzer operable to determine the difference between the output and input.

### CROSS REFERENCE TO RELATED APPLICATIONS

This application claims the benefit of U.S. Provisional patent application Ser. No. 62/396,443, filed on Sep. 19, 2016; the entirety of which is hereby incorporated by reference.

### FIELD OF THE INVENTION

The present invention relates generally to a system and method for determining the location and direction of a catheter tip within a human body. In particular, the invention relates to an echolocation system and method for catheter tip placement.

### BACKGROUND OF THE INVENTION

Medical professionals commonly use catheters for gaining access to a target area within the human body. After the catheter tip is properly positioned at the target area, treatments such as chemotherapy, delivery of medication for pain therapy, and delivery of nutrition in patients who cannot swallow, can be administered. However, if the catheter tip is improperly positioned during insertion, or if the catheter tip migrates out of position after insertion, complications may arise, such as risks to patient safety, increased thrombosis, delays in delivery of therapy, catheter malfunction, and additional hospital costs.

The general standard for proper catheter insertion depends on the type of catheter and the type of treatment. For example, peripherally inserted central catheters, commonly referred to as PICC lines, are typically inserted into a vein in the arm and advanced through the venous system towards the superior vena cava. However, because PICC lines are advanced through the venous system to reach the superior vena cava, the PICC line tip may be inadvertently positioned in a non-target area, such as the internal jugular or subclavian vein. Further, even if a PICC line is properly inserted, the catheter tip could later shift out of position. Therefore, being able to determine the direction and approximate location of the PICC tip is essential for patient safety.

Catheter tip location techniques have improved the ability of medical professionals to verify the location of the catheter tip. One technique involves using an audio sensor that detects an audio signal and transmits it to a processor. The processor determines whether the audio signal corresponds to a target location of the catheter tip. Another technique uses electromagnetic detection and a stylet having an electromagnetic sensor placed inside the lumen of the catheter tip. Electromagnetic systems use an external device positioned directly over the internal target area for generating a magnetic field outside of the body. The electromagnetic sensor on the stylet is then inserted into the body through the catheter lumen and measures when the magnetic flux is at its greatest. A monitor indicates to the user when the electromagnetic sensor on the stylet is centered underneath the external device. Other techniques use ultrasound to determine catheter tip location.

However, the systems and methods described above have deficiencies. For example, interpreting the image using ultrasound can be difficult. Electromagnetic detection may have measurement accuracy disrupted by electromagnetic interference. Additionally, electromagnetic sensing technology may be cost prohibitive.

Therefore, what is needed is a new system and method of locating a catheter tip within a human body that minimize the deficiencies noted above.

### BRIEF SUMMARY OF THE INVENTION

The present invention addresses the deficiencies outlined above. In one aspect of the invention, a system for locating the tip of a catheter inside a human body is provided. The system includes a waveform generator for generating waveforms, a sealed chamber including an emitter configured to emit waveforms generated by and received from the waveform generator, a peripherally inserted central catheter having a saline column for propagating the waveforms toward the heart of a patient, a sound sensor housed within the chamber for sensing sound waves reflected from the heart as a PICC is progressed toward a heart of a patient for placement in the superior vena cava; and a waveform analyzer operable to determine the difference between the output and input.

In another aspect of the invention the tip of the PICC may be fitted with the sound sensor. In such a case, the sound sensor is operably coupled to the waveform analyzer.

In another aspect of the invention the tip of the PICC may be fitted with the sound emitter. In such a case, the sound emitter is operably coupled to the waveform generator.

In other aspects of the invention, multiple sound sensors and multiple sound emitters may be utilized.

### BRIEF DESCRIPTION OF THE DRAWINGS

For a better understanding of the invention, and to show how the same may be carried into effect, reference will now be made, by way of example, to the accompanying drawings, in which:

FIG. 1 depicts the system of the present invention coupled to a catheter that is being advanced towards the superior vena cava.

### DETAILED DESCRIPTION OF THE INVENTION

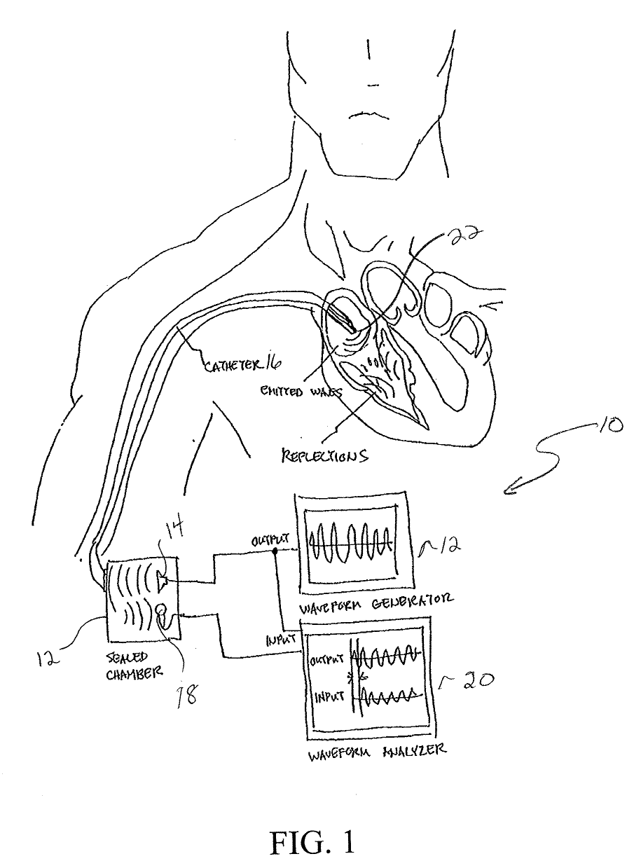

FIG. 1 depicts one aspect of the system 10 in accordance with the invention. Those of skill in the art will appreciate that variations may be made without departing from the broad inventive concept disclosed herein.

In a first aspect of the invention, the system 10 broadly includes a waveform generator 12 for generating waveforms, a sealed chamber 12 including an emitter 14 configured to emit waveforms generated by and received from the waveform generator 22 toward the heart of a patient, a peripherally inserted central catheter 16 having a saline column for propagating the waveforms toward the heart of a patient, a sound sensor 18 housed within the chamber 12 for sensing sound waves reflected from the heart as a PICC is progressed toward a heart of a patient for placement in the superior vena cava; and a waveform analyzer 20 operably coupled and configured to receive input from the sound sensor 18. The waveform analyzer is also operably coupled to the waveform generator 12 and is operable to determine the difference between the output of the waveform generator and the input from the sound sensor. The sound sensor 18 may comprise, by way of example, a microphone, pressure sensor or other types of sensors capable of sensing sound and known to those of skill in the art. In a first aspect of the invention, the sound emitter 14 emits sound waves that propagate in the saline column of catheter 16 toward the tip 22 of the catheter. Any sound that is reflected back from the heart propagates down the saline column and is sensed by sound sensor 18. As the PICC is progressed toward the superior vena cava the magnitude of the sound increases. A drop or decrease in the magnitude of the sound may indicate that the PICC has inadvertently been positioned in a non-target area or has shifted out of position or has been obstructed.

As depicted in FIG. 1, the emitter 14 and sound sensor 18 are positioned within a housing 12. Housing 12 may be fluidly coupled to the saline column of catheter 20 and is desirably water resistant or water proof. Housing 12 may be filled with any media capable of propagating sound pressure, which is generated by the waveform generator and emitted by emitter 14 and sensed by the sound sensor 18, for example saline.

Waveform generator 12 and waveform analyzer 20 each have displays for displaying a graphical form of the sound waves to a user. In operation, waveform generator generates a fixed waveform at a particular frequency and having particular characteristics. The output of waveform generator is operably coupled to and received by emitter 18 and waveform analyzer 20. Emitter transmits the waveform received by the waveform generator through the housing media and through the saline column of the PICC where it is emitted from the tip of the PICC toward the heart. Waveforms are reflected (or echoed) back from the heart and propagate down the saline column into housing 12 and sensed by sound sensor 18 which tracks the reflected waveforms. The input of the sound sensor is transmitted to waveform analyzer 20. The waveform analyzer 20 is capable of displaying a graphical form of the sound waves and determines the difference between the output and input for delay, shifts, distortion and other characteristics.

It will be appreciated by those skilled in the art that changes could be made to the embodiment described above without departing from the broad inventive concept thereof. For example, the sound emitter may be operably coupled to the tip of the PICC and operably coupled to the waveform in generator, wirelessly or tethered. The sound emitter may also be placed at various locations on the body of the patient. Similarly, the sound sensor may be positioned on the tip of the PICC and operably coupled to the waveform analyzer, wirelessly or tethered. The sound sensor may also be positioned at various locations on the body of the patient. In addition, multiple sensors and multiple emitters may be utilized. It is understood, therefore, that this invention is not limited to the particular embodiment disclosed, but it is intended to cover modifications within the spirit and scope of the present invention as defined by the appended claims.

What is claimed is:

1. A system for locating the tip of a catheter inside a human body comprising:
a housing;
a sound emitter and a sound sensor housed in the housing;
a waveform generator configured to generate a fixed waveform at a desired frequency and having desired characteristics and output the fixed waveform to the sound emitter;
a peripherally inserted central catheter fluidly coupled to the housing and configured to propagate the fixed waveform toward a heart of a patient;
a sound sensor housed within said housing, said sound configured to sense sound waves reflected from the heart as the peripherally inserted central catheter is progressed toward a heart of a patient; and
a waveform analyzer operably coupled to the output of the waveform generator and input from the sound sensor, said waveform analyzer operable to determine the difference between the output and input.

2. The system of claim 1 wherein the waveform generator and the waveform analyzer further comprises a graphical display for graphically displaying the wave form.

3. The system of claim 1 wherein said housing contains saline for propagating the fixed waveform generated by the waveform generator and emitted from the sound emitter.
